(19) United States
(12) Patent Application Publication
(10) Pub. No.: US 2008/0274545 A1
(43) Pub. Date: Nov. 6, 2008

(54) Bioreactor Chamber Apparatus, and Method and System for Fabricating and Mechanically Stimulating Natural and Engineered Tissues

(75) Inventors: Catherine K. Kuo, Boston, MA (US); Rocky S. Tuan, Bethesda, MD (US); Mard A. Winslow, Carver, MN (US)

Correspondence Address: EDWARDS ANGELL PALMER & DODGE LLP, PO BOX 55874, BOSTON, MA, 02205, US

(73) Assignee: Government of the US, as represented by the Secret, Rockville, MD (US)

(21) Appl. No.: 11/885,488

(22) PCT Filed: Mar. 2, 2006
(86) PCT No.: PCT/US2006/007664

§ 371 Date: Jun. 3, 2008

Related U.S. Application Data
(60) Provisional application No. 60/658,286, filed on Mar. 2, 2005.

(51) Int. Cl. C12N 5/02; C12M 3/00

(52) U.S. Cl. 435/395; 435/289.1; 435/292.1

(57) ABSTRACT

A bioreactor chamber assembly including a bioreactor chamber and an outer chamber assembly is provided. In one design, the bioreactor chamber includes an upper grip assembly having a plurality of struts extending downward terminating in upper grips, each strut terminating in an upper grip, and a lower grip assembly containing one or more sample compartments and a lower grip. A sample can be formed in situ via injection into a sleeve contained within the sample compartment, where the sleeve encloses the upper and lower grips and enables a construct to form attached to these grips. In a second design, a split mold is received in the bioreactor chamber, the split mold having a cavity for receiving the upper and lower grips. Tissue explants and engineered constructs can be held within these grips. Medium level in the sample compartment is controlled, and different lengths of samples can be accommodated in the sample compartment. The height of the upper grip is adjustable by raising or lowering an extension rod that passes through a dynamic seal to the environment outside the chamber. The bioreactor chamber sample compartment includes a window to permit visualization of the sample as well as light-mediated transformation of biomaterials within the sample compartment. The samples held by grips within these chambers can be subject to uniaxial mechanical stimulation. Medium is perfused around the samples, and medium may be sampled via access ports.

### CROSS-REFERENCE TO RELATED APPLICATION

This application claims the benefit of U.S. Provisional Application Ser. No. 60/658,256 filed on Mar. 2, 2005, the teachings of which are incorporated herein by reference.

### FIELD OF INVENTION

The present invention relates to a mechanoactive bioreactor chamber apparatus, and methods and systems for fabricating one or more samples and applying mechanical stimulation to the fabricated samples in a bioreactor chamber, and more particularly relates to methods and systems for casting one or more cell-seeded tissue-engineered constructs directly in the bioreactor chamber, without requiring a separate seeding step or sample placement between the grips.

### BACKGROUND OF THE INVENTION

Diseased or damaged musculoskeletal tissues are often replaced by an artificial material, cadaver tissue, or donated, allogenic tissue. Tissue engineering offers an attractive alternative whereby a live, natural tissue is generated from a construct made up of a patient's own cells or an acceptable/compatible cell source in combination with a biodegradable scaffold for replacement of defective tissue.

Bioreactors are commonly used to provide a culture environment for developing tissue constructs using biologics and materials such as cells and scaffolds. Conventional bioreactors or bioreactor chambers are configured to receive one sample, and provide mechanical stimulation to the sample, thereby stimulating the development and growth of the sample. Such conventional bioreactors or bioreactor chambers generally utilize medium with a volume of greater than 100 mL.

It would be desirable to provide a bioreactor chamber for accommodating smaller volumes of medium, and capable of simultaneously growing more than one sample. It would also be desirable to enable casting or fabrication of the tissue constructs directly in the bioreactor chamber without requiring a separate cell-seeding step or sample placement between grips.

### SUMMARY OF THE INVENTION

A bioreactor chamber assembly, a bioreactor chamber, and a method for forming cell-seeded constructs directly in the bioreactor chamber are disclosed. The bioreactor chamber assembly preferably includes at least a bioreactor chamber and an enclosure assembly. In particular, the bioreactor chamber can include an upper grip assembly, a lower grip assembly, an extension rod for connecting one or more grips to an actuator located outside the chamber, a clamp to prevent grip motion when the chamber is not connected to the actuator, and a dynamic seal where the extension rod penetrates the chamber environment. The bioreactor chamber is configured to accommodate one or more samples in individual compartments containing a relatively small volume of medium of less than about 10 mL in each sample compartment, with the capability to accommodate greater medium volumes. Alternatively, one or more samples can be accommodated in a shared compartment.

The samples contained within the bioreactor chamber include at least one of tissue explants and tissue engineered constructs made from non-gel or gel scaffolds or combinations thereof. The tissue engineered constructs made from gels or hydrogels can be fabricated from liquid polymer-cell suspensions that form a solid gel after being cast into a mold. The bioreactor chamber preferably includes one or more removable molds or sleeves, one mold contained in each sample compartment for casting of the liquid polymer-cell suspension, thereby allowing each construct to be fabricated directly in the bioreactor chamber, for example, between upper and lower grips. Therefore, when using this method of gel or hydrogel fabrication, it is not necessary to perform a separate cell-seeding step, or a step to place the sample between grips, as required in conventional bioreactors.

The bioreactor chamber can be enclosed by the enclosure assembly to maintain one or more samples and the interior of the bioreactor chamber in a sterile environment. The bioreactor chamber assembly can be placed in an incubator during culture and transported to a device where one or more samples can undergo controlled mechanical stimulation or characterization of materials properties. A clamp preferably is employed for fixing the position of the extension rod with respect to the chamber for the purpose of maintaining the sample height or distance between the upper and lower grips. This permits activities including but not limited to: chamber assembly, sample fabrication, transport during tissue culture, etc. when the chamber and extension rod are not coupled to a mechanical stimulator or characterization device.

Each sample compartment of the bioreactor chamber is configured to allow a mold or sleeve to be installed therein for containing a sample. According to a first preferred embodiment, the upper grip assembly is combined with the lower grip assembly whereby one or more struts from the upper grip assembly are received by one or more sample compartments, with each of the struts terminating in an upper grip. One or more lower grips are affixed to a base of each sample compartment. After a sample is injected into and formed in a sleeve in the sample compartment, the construct attaches to the upper and lower grips, which are preferably wires made of stainless steel or polypropylene, but can be of any other material or form to which the construct can attach or be gripped. After fabrication of a construct, the sleeve can be removed from the sample compartment, for example, by sliding the sleeve up the strut, without disturbing the sample or moving the upper grip assembly. The sleeve for receiving the sample can be removed and discarded after use. The sleeve preferably is made of KYNAR (polyvinylidene fluoride) or another material that is non-adherent for the gel. The distance between the upper and lower grips is adjustable by moving the extension rod, which is preferably attached to the upper grip.

Alternatively, according to a second preferred embodiment of the subject invention, an extension rod terminates in an upper grip, and a lower grip assembly terminates in a lower grip, where the upper and lower grips are received by the sample compartment in the main body of the bioreactor chamber. The main body of the bioreactor chamber can include one or more sample compartments each having an extension rod and upper and lower grips. A split mold is provided with a sample cavity and is configured to be received in the sample compartment, and is capable of receiving the upper and lower grips. The sample can be fabricated in the sample cavity. After sample fabrication, the split mold can be removed from the sample compartment. The enclosure assembly is preferably installed to enclose the sample compartment and maintain the interior of the bioreactor chamber, including the sample compartment, in a sterile environment.

Each sample compartment includes one or more cross ports for maintaining the volume of medium in the sample compartment. When a plurality of cross ports is included, plugging one or more of the lower cross ports enables larger volumes of medium to be contained in a sample compartment, and a longer sample can be accommodated therein.

Other aspects and embodiments of the invention are discussed below.

### BRIEF DESCRIPTION OF THE DRAWING

For a fuller understanding of the nature and desired objects of the present invention, reference is made to the following detailed description taken in conjunction with the accompanying drawing figures wherein like reference character denote corresponding parts throughout the several views and wherein:

FIG. 1 is a perspective view of a bioreactor chamber assembly having a bioreactor chamber according to a first preferred embodiment of the subject invention, and an enclosure assembly with enclosure tube removed for visualization;

FIG. 2 is a perspective view of the bioreactor chamber assembly of FIG. 1, where the enclosure tube of the enclosure assembly is shown enclosing portions of the bioreactor chamber;

FIG. 3 is a top view of the bioreactor chamber assembly of FIG. 1, where the bioreactor chamber assembly has been rotated such that the support columns are aligned laterally;

FIG. 4 is a cross-sectional side view of the bioreactor chamber assembly of FIG. 3 taken along the line IV-IV;

FIG. 5 is a perspective view of the bioreactor chamber of FIG. 1 with enclosure assembly removed for depicting a sample mold or sleeve useful in the subject invention;

FIG. 6 is a top view of the bioreactor chamber of FIG. 5;

FIG. 7 is a cross-sectional side view of the bioreactor chamber of FIG. 6, where the bioreactor chamber has been rotated such that the cross-section is taken through two sample compartments taken along the line VII-VII;

FIG. 8 is an enlarged perspective view of a lower grip assembly of the bioreactor chamber of FIG. 1 showing details of the sample compartment with an inner window in place;

FIG. 9 is an enlarged perspective view of a lower grip assembly of the bioreactor chamber of FIG. 1 with the inner window removed, thereby illustrating a lower grip for accommodating a tissue construct;

FIG. 10 is a perspective view of a bioreactor chamber according to a second preferred embodiment of the subject invention with sample compartment enclosure plates (windows) removed and a split mold installed;

FIG. 11 is a perspective view of the bioreactor chamber of FIG. 10 with the split mold removed and enclosure plates installed;

FIG. 12 is a perspective view of the bioreactor chamber of FIG. 10 with both the split mold removed and enclosure plates removed;

FIG. 13 is a cross-sectional front view of the bioreactor chamber of FIG. 10;

FIG. 14 is a cross-sectional side view of the bioreactor chamber of FIG. 10, in which the bioreactor chamber has been rotated 90° as compared to the view of FIG. 13;

FIG. 15A is an exploded parts view of a split mold useful in the bioreactor chamber of FIG. 10;

FIG. 15B is a perspective view of the split mold depicted in FIG. 15A;

FIG. 16A is a perspective view of the bioreactor chamber according to the second embodiment for multiple samples; and

FIG. 16B is a perspective view of the bioreactor chamber of FIG. 16A in which the split molds are removed from their respective enclosures, and windows cover the enclosures.

### DESCRIPTION OF THE PREFERRED EMBODIMENT(S)

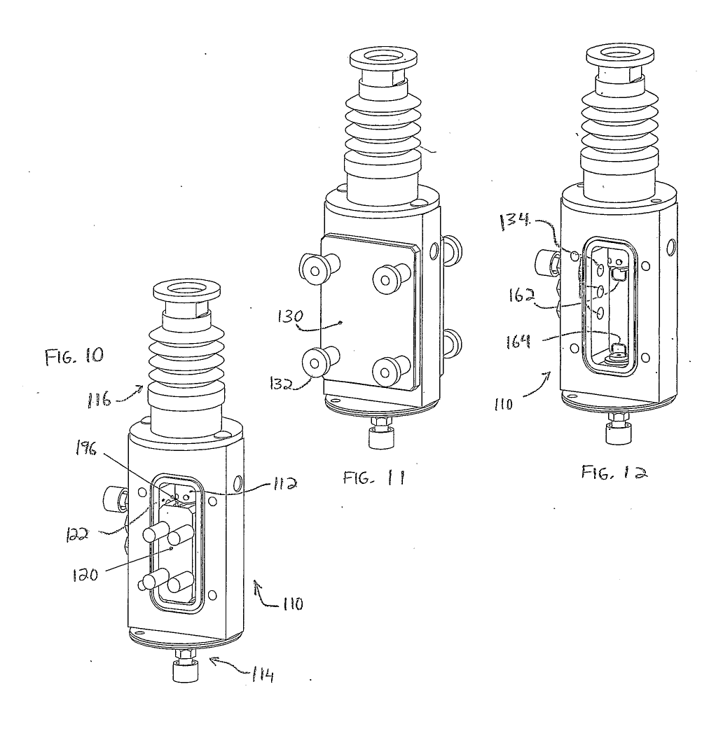

Referring now to the various figures of the drawing wherein like reference characters refer to like parts, there is shown in FIGS. 2 and 11 a bioreactor chamber assembly according to the subject invention, where the bioreactor chamber assembly preferably includes at least a bioreactor chamber and an enclosure assembly. Suitable bioreactor chambers for use in the subject invention are depicted in FIGS. 5 and 10, respectively, although other configurations of bioreactor chambers are within the scope of the subject invention.

The bioreactor chamber itself serves to contain one or more samples, while the enclosure assembly serves to provide an enclosed sterile environment for the interior of the bioreactor chamber. Components of the bioreactor chamber assembly preferably are constructed of materials that are compatible with live cells and compatible with autoclave or gas sterilization.

The bioreactor chamber is configured and arranged to accommodate one or more samples, where each sample is bathed in a relatively small volume of medium of less than about 10 mL, as compared to conventional bioreactors or bioreactor chambers, which generally house a single sample contained in a large volume of medium, generally between 100 and 1000 mL or more. The bioreactor chamber can include one or more sample compartments, each sample compartment preferably capable of holding one sample. For example, in a bioreactor chamber having four sample compartments, it is possible to culture and mechanically stimulate up to four samples simultaneously.

The samples contained within the bioreactor chamber include at least one of tissue explants and tissue engineered constructs. For example, the samples can be, but are not limited to, cell-seeded constructs such as gels, foams, sponges, woven scaffolds, non-woven scaffolds, and braided scaffolds. The tissue engineered constructs may be fabricated from liquid polymer-cell suspensions that form a solid gel after being cast into a mold or sleeve. Alternatively, a non-gel scaffold can be placed in the sample compartment between the appropriate grips and subsequently seeded with cells by filling the sample compartment with medium containing cells. This would allow scaffolds to be seeded with cells in a significantly smaller volume than that of the sample compartment. The bioreactor chamber preferably includes one or more removable molds or sleeves, where the molds or sleeves can include any suitable structure for holding liquid polymer-cell suspensions, which can be removed from the bioreactor chamber. According to the subject invention, one mold is contained in each sample compartment for casting of the liquid polymer-cell suspension, thereby allowing each construct to be fabricated directly in the bioreactor chamber, for example, between upper and lower grips. Therefore, with this technique, it is not necessary to perform a separate cell-seeding step, or a step to place the sample between grips, as required in conventional bioreactors.

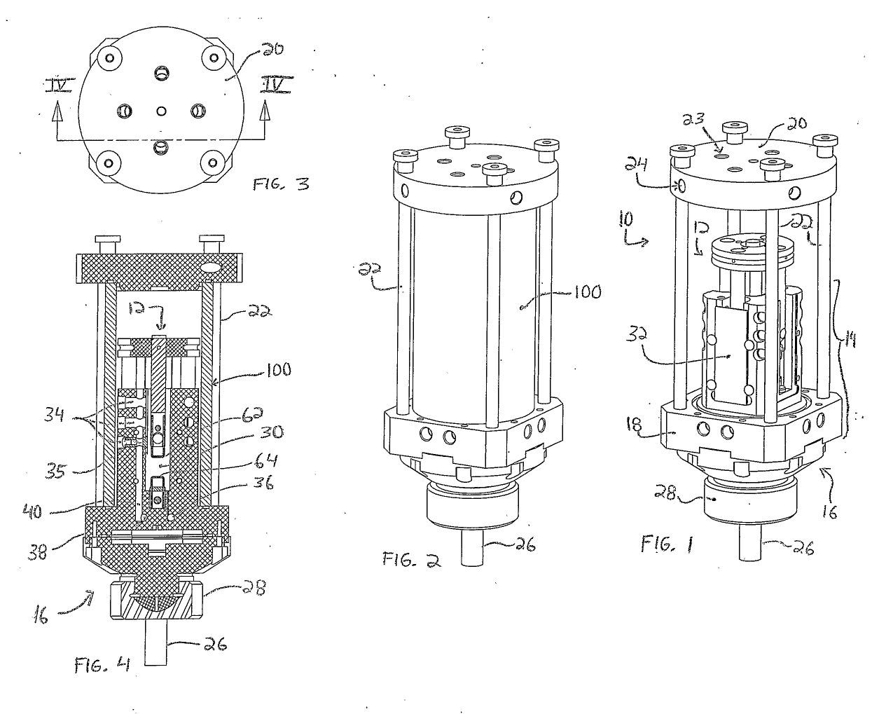

Referring to FIG. 1, a bioreactor chamber assembly 10 according to the subject invention includes a bioreactor chamber and an enclosure assembly, where an enclosure tube is removed in FIG. 1. In particular, the bioreactor chamber according to a first preferred embodiment of the subject invention includes an upper grip assembly 12, a lower grip assembly 14, and an extension rod clamp and dynamic seal assembly 16. The lower grip assembly 14 preferably includes a base 18 for receiving one or more components of the enclosure assembly, and can include holes for receiving lid support columns 22. As shown in FIG. 1, the enclosure assembly has at least a chamber lid 20 and a plurality of lid support columns 22 attached to the chamber lid. Although four lid support columns 22 are depicted in FIG. 1, a particular enclosure assembly may include any suitable number of lid support columns.

Referring to FIG. 2, the enclosure assembly also can include an enclosure tube 100 circumferentially arranged inside the lid support columns 22. The enclosure tube 100 can be cylindrical in shape and is sized lengthwise to fit between the chamber lid 20 and the base 18, thereby enclosing exposed portions of the upper and lower grip assemblies 12 and 14 (see FIG. 1). Although the enclosure tube 100 is intentionally omitted from FIG. 1 for convenience, it is clearly depicted in FIG. 2, as the enclosure tube 100 is enclosed by the lid support columns 22. The enclosure tube depicted in FIG. 2 is preferably circular in cross-section, and is preferably transparent. The enclosure tube can be made of a strong, durable transparent material, such as certain types of glass or plastic, thereby enabling viewing of the bioreactor chamber contained therein. More preferably, the enclosure tube 100 is made of PYREX. When installed in the bioreactor chamber assembly, the enclosure tube 100 can enclose portions of the upper and lower grip assemblies 12 and 14, thereby maintaining a sterile environment.

The chamber lid 20 optionally can include one or more overhead ports 23 for accessing the bioreactor chamber from above, for example, to enable external connections for transducers and sensors used to monitor the growth and development and/or characterize samples in the bioreactor chamber. The chamber lid 20 also can include one or more side ports 24 for connecting to other equipment such as air filters or sensors.

Although not necessary to the functioning of the bioreactor chamber itself, the chamber lid 20, support columns 22, and enclosure tube 100 serve to house and protect the upper and lower grip assemblies 12 and 14, such that the bioreactor chamber assembly 10 functions as a stand-alone or modular unit. The enclosure tube 100, chamber lid 20, and support columns 22 are removable to provide access to samples contained in the bioreactor chamber, without disturbing the environment of the samples held by the grips. The bioreactor chamber assembly 10 can be maintained in an incubator (not shown) or water bath (not shown) during culture and subsequently transferred to a mechanical stimulator (not shown) or mechanical test device (not shown) or other device (not shown) for stimulating or characterizing the samples. Various wires, tubes, or other conduits can be threaded through the overhead ports 23 or side ports 24 as needed.

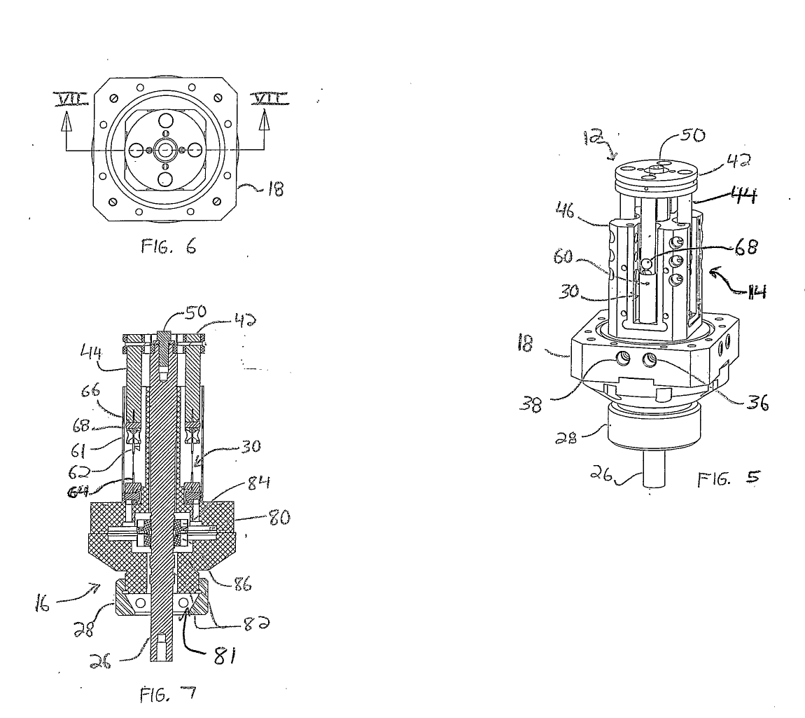

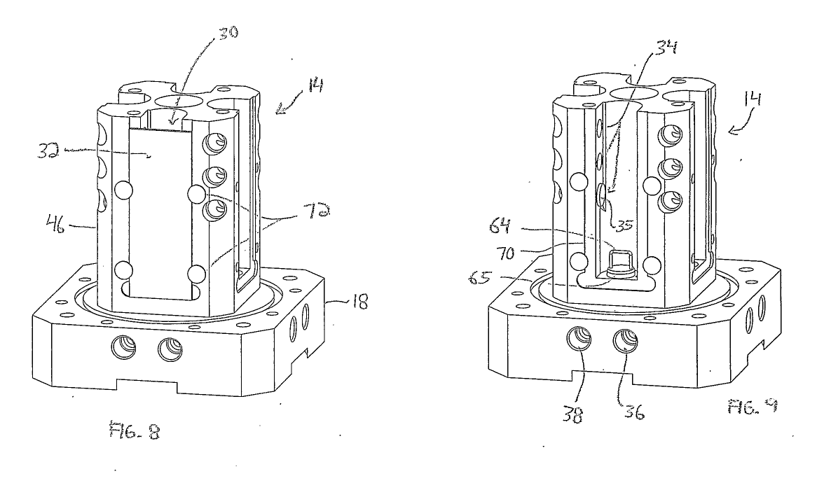

As shown in FIGS. 1 and 4, the extension rod clamp and dynamic seal assembly 16 includes a hand nut 28, and other components, to be described in greater detail below with respect to FIG. 7. The clamp and dynamic seal assembly 16 is assembled around an extension rod 26 extending longitudinally through approximately a center of the bioreactor chamber, where the extension rod 26 forms part of the upper grip assembly 12. The extension rod 26 is preferably a cylindrical shaft as shown in FIG. 1, and configured for coupling to an actuator (not shown) or insertion into a stand (not shown) to support the bioreactor chamber assembly 10. For example, the stand can be a stationary block which supports the assembly for stabilization purposes while performing work on sample(s) contained in the bioreactor chamber. Alternatively, the stand can be omitted as desired. The bioreactor chamber assembly is configured as a module that can be placed in an incubator, a water bath, a mechanical testing device, a mechanical stimulator, or another machine or device for performing work on the sample(s). A suitable mechanical testing device for use with the bioreactor chamber assembly is the ELECTROFORCE 3200 (or ELF) sold by EnduraTEC Systems Corporation of Minnetonka, Minn. A suitable mechanical stimulator is the DynaGen TC-20 sold by Tissue Growth Technologies Corporation of Minnetonka, Minn. The ELF or DynaGen systems can be used to apply mechanical stimulation to cell-seeded constructs in the bioreactor chamber. Mechanical forces are applied as uniaxial tensile or compressive forces, or uniaxial specimen elongation or compression. The forces and displacements may be static or dynamic in nature, and of varied magnitude, frequency, duration, and wave form.

Controlled motion or work on the sample is generated with relative motion or force applied to the upper or lower grips or both. For example, preferably the lower grips are mechanically restrained while motion or force is applied to the upper grips through the extension rod and externally coupled actuator (not shown).

Details of a sample compartment are depicted in FIG. 4, which is a cross-sectional view taken through the bioreactor chamber assembly as shown in FIG. 3. In an exemplary sample compartment 30 for holding a sample such as a tissue-engineered construct, the construct can be fabricated from cells suspended in a liquid that form a solid gel after being cast in a mold or sleeve contained in the sample compartment 30. Alternately, a scaffold or other material attached to upper and lower grips within the mold or sleeve can be seeded with cells when the mold or sleeve is filled with medium containing cells. The sample compartment 30 can hold a volume of medium of less than about 10 mL, or preferably about 3 mL to 10 mL of medium. But another bioreactor chamber can be sized differently to hold larger or smaller quantities of medium.

The bioreactor chamber depicted in FIGS. 1 to 9 includes four identical sample compartments arranged in a circular array, each for holding a separate sample, and thus the bioreactor chamber can accommodate up to four samples simultaneously, although other bioreactor chambers can be formed with any number of sample compartments in a circular or rectangular array or other arrangement. In the exemplary bioreactor chamber of FIG. 4, the sample compartment 30 can hold up to about 10 mL of medium. The volume of medium contained in an individual sample compartment 30 is controlled by a plurality of cross ports 34.

Each sample compartment 30 of the bioreactor chamber depicted in FIG. 4 includes three cross ports 34 at varied elevation for controlling volume of medium, but any number of cross ports can be provided. According to a first preferred embodiment of the subject invention, it is possible to control the volume of medium contained in the sample compartment 30 by plugging one or more of the cross ports 34. For example, as shown in FIG. 4, a lower cross port is plugged by inserting a plug 35 in the cross port 34. The enlarged view of FIG. 9 shows the plug 35 in blocking engagement with the lower cross port 34, while the middle and upper cross ports remain unplugged. In the bioreactor chamber of the subject invention, by plugging the lower cross port 34, a larger volume of medium can be contained in the sample compartment, thereby accommodating a longer sample.

Medium enters the bioreactor chamber through a medium inlet port 36 in the lower grip assembly 14, where the medium inlet port 36 is positioned below the sample compartment 30, as shown in FIGS. 4, 5, 8, and 9. Medium can exit the bioreactor chamber through a medium outlet port 38 (see FIGS. 4 and 9). Outlet conduit 40 connects the cross ports 34 and the medium outlet port 38, such that medium escapes the sample compartment 30 by passing through any unplugged cross ports 34, and then through the outlet conduit 40 to the medium outlet port 38, where it exits the bioreactor chamber. Preferably the medium is re-circulated in a closed-loop system, such that oxygen-permeable tubing (not shown) connects the medium outlet port 38 to the medium inlet port 36, either directly or indirectly through a reservoir located between the ports for replenishing the medium. The flow rate and amount of medium is preferably controlled by an external pump or by supervisory computer hardware and/or software that can operate a pump, for example to re-circulate the medium through the bioreactor chamber. In a closed-loop system, medium is added or removed in a sterile manner preferably via injection or aspiration, respectively, through sterile filters in the bioreactor chamber or the bioreactor chamber assembly, or via the reservoir or at any other part of the system. Preferably, the use of oxygen-permeable tubing enables oxygenation of the medium as it circulates through the closed-loop system.

The bioreactor chamber assembly depicted in FIG. 2 also allows for closed-loop control of nutrient and bioactive factor(s) perfusion, as well as temperature and CO₂ levels and other gas levels. For each sample compartment, nutrient medium is circulated via ports 36 and 38 as described. Bioactive factor(s) may be delivered into the sample compartment 30 via ports 23 or through the circulated medium via port 36. CO₂ and other gases may be regulated via advection through ports 23 and 24. Temperature may be regulated by placing the device in an incubator or water bath or other device that can control temperature.

The bioreactor chamber according to the first preferred embodiment of the subject invention is shown in greater detail in FIGS. 5 and 7. Referring to FIG. 5, the upper grip assembly 12 of the bioreactor chamber includes the extension rod 26, an upper carousel 42 generally shaped as a disc, and a plurality of struts 44 extending downwardly from the upper carousel 42, the struts configured to fit within the sample compartments 30, where one strut 44 is provided for each sample compartment 30. The upper carousel 42 and struts 44 are configured to be received in a chamber body 46 of the lower grip assembly 14, where the chamber body 46 includes the sample compartments 30 and other components for holding medium in the sample compartments. The chamber body 46 preferably is fixed to the base 18, although the chamber body and base can be provided as separate components if desired.

As shown in FIGS. 5 and 7, the upper carousel 42 preferably is fixed to the extension rod 26 by a screw 50. The extension rod 26 extends longitudinally through the chamber body 46 of the lower grip assembly 14 and can be locked into place by the clamp shown in FIG. 7. Referring to FIG. 7, the hand nut 28 can be rotated to drive an annular wedge 81 uniformly against the extension rod 26 and thus lock the position of the upper grip assembly 12 with respect to the lower grip assembly 14. Conversely, loosening the hand nut 28 relaxes the wedge and the extension rod 26 is free to move longitudinally. The dynamic seal 80 is preferably a diaphragm-type membrane seal and is therefore preferably non-sliding and without friction. The dynamic seal preferably constitutes a flexible material such as KYNAR (polyvinylidene fluoride) or silicone that is sandwiched between an internal collar 84 and an external collar 86, which are preferably forced into mating engagement by counter rotating them as they engage threads along the extension rod.

Preferably the dynamic seal 80 forms a seal around the extension rod 26 and at its outer perimeter is sandwiched between the chamber base 18 and clamp assembly 16. This arrangement enables extension rod motion without leakage and without compromising the sterile environment of the interior of the bioreactor chamber. The extension rod 26 also includes a plurality of threaded and smooth sections 82 arranged along its length near the dynamic seal 80 and corresponding to different positions for the internal and external collar 84, 86 and dynamic seal 80. Positioning the collars and dynamic seal along the extension rod defines the distance between the upper and lower grips. As the extension rod 26 is fixed to the upper carousel 42, by adjusting the height of the extension rod 26, the struts 44 attached to the upper carousel 42 are raised or lowered, thereby raising or lowering the upper grip.

Referring to FIG. 5, to fabricate a gel-based construct, a liquid polymer-cell suspension is cast in a mold or sleeve 60 contained within the sample compartment 30. The mold or sleeve can be any structure capable of holding a liquid cell-gel suspension, where the mold or sleeve is configured to be removable from the sample within the bioreactor chamber, or removable from both the sample and the bioreactor chamber. For example, the sleeve 60 or an equivalent structure is suitable for use in the embodiment depicted in FIG. 5. The terms “mold” and “sleeve” are used interchangeably in the subject application, and are not meant to limit the type of structure for holding a sample.

A sample made up of cells suspended in a liquid polymer can be introduced into the sleeve 60 by a syringe or thin pipette, where the syringe can be received through a port 61 arranged in the strut 44 (see FIG. 7). The sleeve can provide a leak-proof seal around the lower grip without the aid of conventional seals such as o-rings.

The sleeve 60 functions as a mold for receiving the sample and allowing the gel to set between the grips, after which the sleeve is moved away from the sample by sliding it longitudinally over the strut 44. The removed sleeve can be attached to an upper portion of the strut by a pin or screw while the sample remains in the sample compartment 30. Alternatively, the sleeve 60 can be cut away and removed from the bioreactor chamber. Preferably the sleeve 60 is removed from the sample compartment 30 after the gel has set between the upper and lower grips, 62 and 64, respectively, and thereafter the sample compartment 30 is filled with medium.

Sleeves useful in the subject invention can be made of KYNAR or another material, such that the sleeves preferably are removed from the sample compartment after use and discarded. The sleeves also preferably are transparent to enable viewing of the tissue constructs during fabrication, and provide for alternative methods of solidifying the gel including electromagnetic radiation induced setting of the gel or hydrogel, such as ultraviolet radiation-induced radical polymerization.

As shown in FIG. 5, the sleeve 60 preferably encloses at least one grip, and more preferably encloses upper and lower grips, 62 and 64, respectively. FIG. 9 depicts an enlarged view of the lower grip 64, where the lower grip 64 is attached to a lower strut 65 affixed to the base of the sample compartment 30. The upper grip 62 is connected to the strut 44 and includes a linear extension 66 extending into the strut 44, where the linear extension 66 is fixedly attached to the strut 44 by one or more screws or pegs 68 as shown in FIGS. 5 and 7. The position and height of the upper grip 62 can be varied depending upon the sample size by adjusting the position of the extension rod 26, as described above. A similar arrangement can be provided for the lower grip 64, where a linear extension of the lower grip preferably extends into the lower strut 65 and is fixedly attached by one or more screws or pegs.

Both the upper and lower grips 62 and 64, and their respective linear extensions, preferably are square or round wires of an appropriate gauge made of stainless steel or other materials such as polypropylene, or other materials to which a construct including cells on or in a gel will attach. The upper and lower grips 62 and 64 are firmly supported within their respective struts using screws or pegs. Each of the upper and lower grips are easily substitutable by loosening or removing the screws or pegs, and replacing the wires. The type of grips used can also be changed simply by exchanging the struts 44 and 65 with struts that are outfitted with an alternative grip form, such as clamps to grab a tissue explant or different type of tissue construct, e.g., a non-gel. Instead of the wire grips described herein, the upper and lower grips can constitute, but are not limited to, wire or plastic mesh, sponge, clamps, or alligator clamps into which the gels can set and lock or attach, or to grab and hold the tissue constructs/gels. The grips can also serve to grab other scaffolds to which a construct comprised of cells on or in a gel can attach. Alternatively, the grips can be replaced by compression plates having a flat surface to enable compression of the sample within the sample compartment, instead of providing tensile stimulation.

In the embodiment of FIGS. 5 and 7, at least three settings are provided to accommodate different sizes of samples. For example, by adjusting the extension rod 26 to a lowermost position, a smaller sample can be contained in the sample compartment. In a middle position, a larger sample can be contained, and in the uppermost position, the largest sample can be contained. For example, the three sample sizes or lengths can be 10 mm, 20 mm, and 30 mm, although other sizes may be appropriate depending on the size of the bioreactor chamber or length of the struts. The number of settings can be less or more than three settings, and the distance between the settings can be greater or less than increments of 10 mm.

As shown in FIGS. 8 and 9, the sample compartment 30 is open at the top for receiving the strut 44. A window 32 preferably covers the sample compartment 30, thereby holding the sample and medium inside the sample compartment. The window 32 preferably is made of glass, such as 1 mm thick borosilicate, and is preferably sealed with silicone grease or conventional rubber gaskets. The window 32 is sized appropriately to fit within a recess 70 of the chamber body, and is secured by one or more screws 72. Alternatively, the window 32 can be removed from one or more sample compartments so that one or more samples can share the same volume of medium.

After a sample, such as cells suspended in a liquid polymer, is cast in a sleeve 60, the sample can form into a solid gel. For example, this transformation occurs when the bioreactor chamber assembly 10 is placed in an incubator for an appropriate amount of time. During this process, the tissue construct sets around the upper and lower grips 62 and 64. The solid gel can form by attaching to the grips such as wires, which are contained within the sleeve 60. Optionally, one or more compounds or molecules can be added to the sample compartment to chemically cure the sample. Thereafter, the bioreactor chamber assembly can be connected to a device for applying controlled mechanical stimulation.

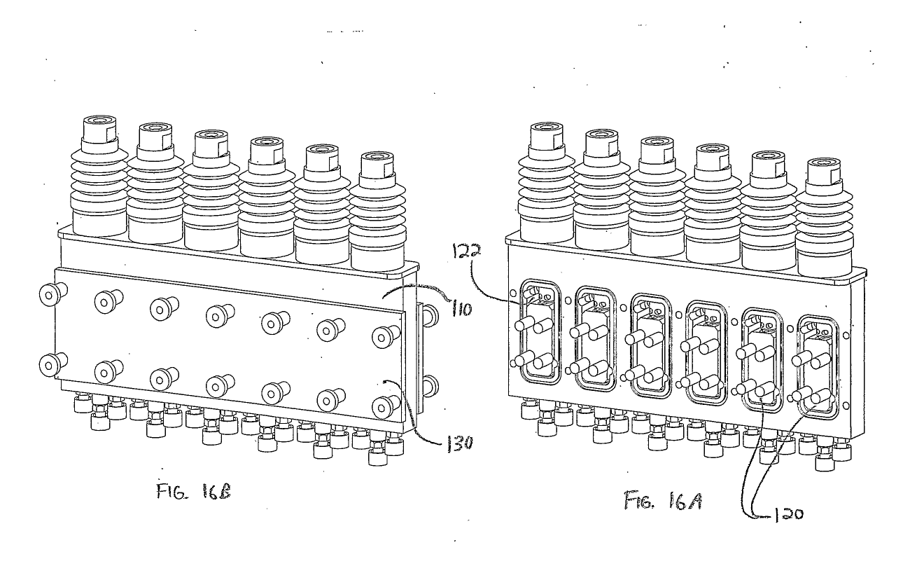

A second preferred embodiment of the bioreactor chamber is depicted in FIGS. 10 to 16B. As shown in FIG. 10, a bioreactor chamber according to the second preferred embodiment includes a main body 110, an extension rod 112 terminating in an upper grip (see FIG. 13), a lower grip assembly 114, a clamp, and a dynamic seal assembly 116. Preferably the main body 110 is formed with a sample compartment 122 that is configured and arranged to receive a removable mold such as a split mold 120. The split mold 120 will be discussed in greater detail with reference to FIGS. 15A-15B. As described herein, the main body 110 includes at least one sample compartment for holding up to about 10 mL of medium. According to the second preferred embodiment, the split mold 120 or a similar removable mold structure is used in place of the sleeve described in the first preferred embodiment for containing a sample.

The bioreactor chamber depicted in FIGS. 10 to 15B includes one sample compartment. However, other configurations of the bioreactor chamber can include a plurality of sample compartments, each sample compartment capable of accommodating a removable mold, such as the split mold, and thereby capable of holding multiple samples. A bioreactor chamber housing multiple samples is within the scope of the second preferred embodiment, and can hold any number of sample compartments. For example, FIGS. 16A and 16B depict a bioreactor chamber capable of holding multiple samples. Alternately, a bioreactor chamber can house a plurality of samples fabricated or seeded with cells in individual molds and subsequently cultured in a single compartment sharing the same volume of medium.

Referring to FIGS. 11 and 12, when the split mold 120 is removed from the sample compartment 122, an enclosure plate 130 can be affixed to at least one face of the main body 110, for example, by using screws, pegs, bolts, or other fasteners. To create a sealed and sterile environment two enclosure plates are required to enclose the sample compartment 122 through the main body. Preferably, the enclosure plates 130 are transparent. The enclosure plates 130 can be made from polycarbonate, borosilicate glass, or quartz glass. As shown in FIG. 11, four fasteners 132 are placed at the four respective corners of the enclosure plates 130, although more or fewer such fasteners may be used. FIG. 12 depicts a state in which both enclosure plates 130 are removed from the main body 110, thereby revealing upper and lower grips 162 and 164 protruding into the sample compartment 122.

Referring to FIGS. 10, 13, and 14, the split mold 120 generally is placed in the main body 110 of the bioreactor chamber during casting, in which cells suspended in a liquid polymer are cast in the split mold 120, and enabled to form into a solid gel. Alternately, medium containing cells can fill the cavity of the split mold to seed a non-gel scaffold with cells within a small volume. After the gel is solid, the split mold 120 can be removed, and the enclosure plates 130 attached to the main body 110 of the bioreactor chamber. Thereafter, with the enclosure plates affixed, activities such as culture and mechanical stimulation can be performed on one or more samples contained within the bioreactor chamber assembly.

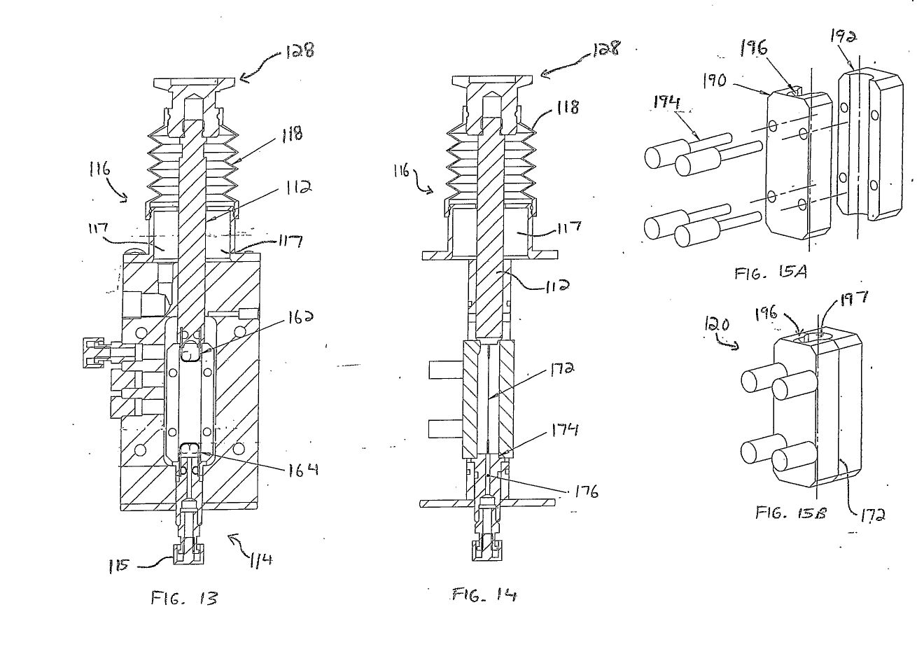

The internal structure of a bioreactor chamber according to the second preferred embodiment is described in greater detail with reference to FIGS. 13 and 14. The extension rod clamp and dynamic seal assembly 116 is assembled around the extension rod 112, which extends longitudinally through a bore located approximately at the center of the bioreactor chamber. A lower end of the extension rod 112 terminates in the upper grip 162 (similar to the upper grip 62 described in the first preferred embodiment). The upper grip 162 preferably is attached to the extension rod 112 by using screws, pegs, or other fasteners.

An upper end of the extension rod 112 terminates in a coupling 128. The coupling 128 can be a mechanical interface to an actuator such as a mechanical stimulator device (not shown) or materials characterization device (not shown). The coupling 128 shown in FIGS. 10 to 14 is a more complex form than that shown for extension rod 26 described in the first preferred embodiment. When coupled to an actuator, the extension rod 112 terminating in the upper grip 162 can apply mechanical motion to the sample to result in changes in the dimensions of the sample. To fix the position of the upper grip 162, a clamp 117 is engaged to fix the extension rod 112 with respect to the main body 110 and the lower grip assembly 114. The clamp 117 may be of the annular wedge type described in the first preferred embodiment, where the clamp 117 preferably surrounds the extension rod 112 and forms a frictional grip. A bellows seal 118 can be clamped or otherwise affixed to a portion of the clamp 117, where the bellows seal 118 preferably is made of silicone or a similar material, and is formed in an accordion-like configuration, such that the bellows seal 118 can collapse in response to longitudinal motion of the coupling 128.

The lower grip 164 is securely attached to an upper end of the lower grip assembly 114 by screws, pegs, or other fasteners. The lower grip assembly 114 preferably extends through a bore at the center of the main body 110 of the bioreactor chamber. In this example, the lower grip assembly 114 is fixed, but alternately an additional coupling (not shown) and bellows seal (not shown) for the lower grip assembly 114 would allow movement of the lower grip. A perfusion port and fitting at the lower end of the lower grip assembly 114 optionally can be sealed by a fitting cap 115 and are discussed in greater detail below.

The upper and lower grips 162 and 164 preferably are made of wire, such as stainless steel wire, but can be of any material or form as described with reference to the first preferred embodiment. The grips are designed to fit within the sample cavity of the split mold 120, and thereby support a sample during casting or cell seeding. The grips 162 and 164 are adjustable to different heights and can be removed or replaced as desired. As described above, the extension rod 112 can be adjusted to a plurality of settings, thereby raising or lowering the upper grip 162 to accommodate different sample sizes.

The split mold 120 is shown in greater detail in FIGS. 15A-15B. The split mold 120 preferably includes first and second mold halves 190 and 192, where the first mold half 190 includes a liquid entry port 196 for introducing a liquid sample or medium containing cells into the closed mold. Preferably a sample made up of cells suspended in a liquid polymer is introduced into the split mold 120 when the mold is in a closed or assembled closed state (see FIG. 15B), for example, by using a syringe or thin pipette. Referring to FIG. 10, the liquid sample or medium containing cells can be introduced when the split mold 120 is placed in the main body 110 of the bioreactor chamber.

As shown in FIG. 15B, when the split mold halves are assembled, the liquid entry port 196 is provided adjacent to a hole 197 which defines a sample cavity extending through the split mold 120. The hole 197 extending through the split mold 120 is appropriately sized to receive the upper and lower grips 162 and 164 as well as a desired initial sample volume for samples fabricated from gels or hydrogels. Referring to FIGS. 13 and 14, the split mold 120 is placed in the main body 110 of the bioreactor chamber, such that the grips 162 and 164 extend into the hole 197 for supporting a sample in the sample compartment of the split mold.

As shown in FIGS. 14 and 15B, in an assembled state the split mold 120 provides a precision fit between the assembled mold halves to prevent leakage of the sample. The mold halves 190 and 192 can be held together by fasteners such as pegs 194, as shown in FIG. 15A or other means as described earlier for the enclosure plate(s) 130. When the split mold 120 is installed in the bioreactor chamber, a precision fit is formed between the hole 197 at the bottom of the split mold 120 and the lower grip assembly 114, thereby preventing leakage. The split mold 120 is installed by inserting one half of the split mold 190 into one open side of the sample compartment 122 and the other half of the split mold 192 into the other open side of the sample compartment 122. The mold halves are inserted until they engage the upper extension rod 112, lower grip assembly 114, and make contact with each other along a parting line 172. Fasteners 194 secure the mold halves. When it is time to remove the split mold, for example after the gel is solid, the fasteners are removed and each mold half is withdrawn from the sample compartment 122.

Medium can be circulated through the bioreactor chamber in a manner similar to the first preferred embodiment. For example, the main body 110 of the bioreactor chamber preferably includes a plurality of cross ports 134, such as the three cross ports depicted in FIG. 12, where one or more of the cross ports 134 can be plugged to control the volume of medium contained in the sample compartment 122, in a manner similar to the cross ports 34 depicted in FIG. 9 of the first preferred embodiment. A lower perfusion port 176 shown in FIG. 14 can be used as an inlet or outlet for closed-loop control of nutrient and bioactive factor(s) perfusion. The lower perfusion port 176 can be plugged during sample fabrication or cell seeding (see FIG. 14).

FIGS. 16A and 16B depict a bioreactor chamber according to the second preferred embodiment which is capable of holding multiple samples, each sample being fabricated or seeded with cells in a separate split mold 120, the split molds being housed in respective sample compartments 122 of the bioreactor chamber. In FIG. 16A, during a casting, fabrication, or cell seeding step, the split molds 120 identical or different in design of the cavity are received in respective sample compartments 122 of the bioreactor chamber, similar to the arrangement depicted in FIG. 10. Instead of providing two halves of a single split mold 120 for individual sample compartments 122, a plurality of split mold halves could be manufactured as a single component or attached to a common support. Subsequently, as shown in FIG. 16B, with the split molds 120 removed from the sample compartments 122, an enclosure plate 130 can be affixed to at least one face of the main body 110, for example by using screws, pegs, bolts, clamps, latches, hinges, or other fasteners as described earlier to attach the enclosure plate 130 to the main body 110. Instead of providing a single enclosure plate 130, multiple enclosure plates can be attached to the main body, for example, to cover one or more of the sample compartments. With the enclosure plate 130 affixed, activities such as culture and mechanical stimulation can be performed on the one or more samples contained within the bioreactor chamber. Alternately, another bioreactor chamber could contain a plurality of samples in a common compartment with a shared volume of medium.

Materials useful in the bioreactor chamber for the cell-seeded constructs can include but are not limited to acellularized tissues, collagen, elastin, gelatin, glycosaminoglycan starch, chitin, chitosan, hyaluronan, alginate, poly(alpha-hydroxy ester)s (such as polylactic acid, polyglycolic acid, and poly(epsilon-caprolactone)), polyanhydrides, polyorthoesters, polyphosphazens, poly(propylene fumarate), polyurethane, polyvinyl alcohol, and other biodegradable and non-biodegradable materials, and combinations thereof. Also useful in the bioreactor of the invention are gels or hydrogels for the tissue engineered construct, which are viscoelastic solids. Such materials can include but are not limited to alginate, chitosan, polyethylene oxide, polyethylene glycol, collagen, hyaluronan, agarose, other natural and synthetic polymers and combinations thereof.

In embodiments where one or more cells are suspended in the polymer, the cells may be any cell or cell type, for instance a prokaryotic cell or a eukaryotic cell. For example, the cell may be a bacterium or other single-cell organism, a plant cell, an insect cell, a fungi cell or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a human cell, a primate cell, a bovine cell, a horse cell, a porcine cell, a goat cell, a dog cell, a cat cell, or a cell from a rodent such as a rat or a mouse. If the cell is from a multicellular organism, the cell may be from any part of the organism. For instance, if the cell is from an animal, the cell may be a cardiac cell, a fibroblast, a keratinocyte, a hepatocyte, a chondrocyte, a neural cell, an osteoblast or osteocyte, a muscle cell, a blood cell, an endothelial cell, an immune cell (e.g., a T-cell, a B-cell, a macrophage, a neutrophil, a basophil, a mast cell, an eosinophil), a stem cell, cartilage cell, somatic stem cell, fibroblasts, fibrocytes, vascular endothelial cells, cartilage cells, liver cells, small intestine epithelial cells, epidermis keratinized cells, osteoblasts, mesenchymal stem cells derived from bone marrow and other adult tissues, embryonic stem cells, etc. In some cases, the cell may be a genetically engineered cell. In certain embodiments, the cell may be a Chinese hamster ovarian (“CHO”) cell or a 3T3 cell. In some embodiments, more than one cell type may be used simultaneously, for example, fibroblasts and hepatocytes. In certain embodiments, cell monolayers, tissue cultures or cellular constructs (e.g., cells located on a nonliving scaffold), and the like may also be used in the polymer. The precise environmental conditions necessary in the polymer for a specific cell type or types may be determined by those of ordinary skill in the art. The cells may be transformed, expressing or over-expressing native or altered forms of proteins, peptides, and/or nucleic acids, or modified to suppress or reduce the expression of specific gene products. The cells may be cells useful for growing on scaffolds for tissue engineering (immature tooth pulp, cartilage, cardiac cells, liver cells, kidney cells, stem cells, and the like), cells for tissue replacement (blood cells, skin cells, and the like), or cells for bioactive factor production.

In some instances, the cells may produce chemical or biological compounds of therapeutic and/or diagnostic interest, for instance, in picogram, nanogram, microgram, milligram or gram or higher quantities. For example, the cells may be able to produce products such as monoclonal antibodies, proteins such as recombinant proteins, amino acids, hormones, vitamins, drug or pharmaceuticals, other therapeutic molecules, artificial chemicals, polymers, tracers such as GFP (“green fluorescent protein”) or luciferase, etc. In one set of embodiments, the cells may be used for drug discovery and/or drug developmental purposes. For instance, the cells may be exposed to an agent suspected of interacting with the cells. Non-limiting examples of such agents include a carcinogenic or mutagenic compound, a synthetic compound, a hormone or hormone analog, a vitamin, a tracer, a drug or a pharmaceutical, a virus, a prion, a bacteria, etc. For example, in one embodiment, the invention may be used in automating cell culture to enable high-throughput processing of monoclonal antibodies and/or other compounds of interest. In another embodiment, the invention may be used to screen cells, cell types, cell growth conditions, or the like, for example, to determine self viability, self production rates, etc. In some cases, the invention may be used in high throughput screening techniques. For example, the invention may be used to assess the effect of one or more selected compounds on cell growth, normal or abnormal biological function of a cell or cell type, expression of a protein or other agent produced by the cell, or the like. The invention may also be used to investigate the effects of various environmental factors on cell growth, cell biological function, production of a cell product, etc.

Although a preferred embodiment of the invention has been described using specific terms, such description is for illustrative purposes only, and it is to be understood that changes and variations may be made without departing from the spirit or scope of the following claims.

### INCORPORATION BY REFERENCE

All patents, published patent applications, and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A bioreactor chamber, comprising:
at least one sample compartment configured to hold a sample;
a strut arranged in the sample compartment, the strut including at least one grip; and
a sleeve enclosing the at least one grip for receiving the sample.

2. The bioreactor chamber of claim 1, further including one or more additional sample compartments for holding additional samples.

3. The bioreactor chamber of claim 1, wherein the strut terminates in an upper grip, and a lower grip is fixed to the sample compartment.

4. The bioreactor chamber of claim 3, wherein the upper and lower grips are wires made from stainless steel.

5. The bioreactor chamber of claim 3, wherein the upper and lower grips are made from polypropylene.

6. The bioreactor chamber of claim 3, wherein the upper and lower grips are made of porous scaffolding selected from the group consisting of sponges, meshes, and woven and non-woven scaffolds.

7. The bioreactor chamber of claim 6, wherein the porous scaffolding is made of biocompatible materials.

8. The bioreactor chamber of claim 3, wherein the upper and lower grips are clamps.

9. The bioreactor chamber of claim 1, wherein the sleeve is configured to slide along the strut.

10. The bioreactor chamber of claim 9, wherein the sleeve is removable from the sample compartment upon setting of the sample.

11. The bioreactor chamber of claim 1, wherein medium is circulated through the sample compartment after removal of the sleeve.

12. The bioreactor chamber of claim 11, wherein the sample compartment includes a window covering the sample compartment to hold the medium in the sample compartment and provide for sample visualization, and light-mediated transformation of biomaterials contained within the sample compartment.

13. The bioreactor chamber of claim 11, wherein the level of medium contained in the sample compartment is controlled by a plurality of ports.

14. The bioreactor chamber of claim 13, wherein at least one of the ports is configured to receive a plug.

15. The bioreactor chamber of claim 13, wherein the medium is recirculated through the bioreactor chamber.

16. The bioreactor chamber of claim 11, wherein the sample compartment is configured to hold up to about 10 mL of medium.

17. The bioreactor chamber of claim 11, wherein the sample compartment is configured to hold between about 3 mL and 10 mL of medium.

18. The bioreactor chamber of claim 1, wherein the strut is adjustable in the sample compartment by raising or lowering an extension rod.

19. The bioreactor chamber of claim 18, wherein raising or lowering the extension rod adjusts the at least one grip.

20. The bioreactor chamber of claim 1, wherein the sample is a tissue-engineered construct.

21. The bioreactor chamber of claim 1, wherein the sample is a tissue explant.

22. The bioreactor chamber of claim 1, wherein the bioreactor chamber is configured to be received in an assembly, the bioreactor chamber being enclosed along its length by a plurality of columns supported by a lid and a base.

23. The bioreactor chamber of claim 22, and further including an outer tube or enclosure tube positioned inside the plurality of columns for maintaining the bioreactor chamber in a sterile environment.

24. The bioreactor chamber of claim 22, wherein the bioreactor chamber enclosed in the assembly is a module for being received in an incubator, water bath, or a mechanical testing device.

25. A bioreactor chamber, comprising:
a plurality of sample compartments configured to hold one or more samples;
a plurality of struts arranged in the sample compartments, each strut including at least one grip; and
a sleeve enclosing the at least one grip for receiving each sample.

26. A bioreactor chamber, comprising:
an upper grip assembly including a carousel and a plurality of struts extending from the carousel, where each strut terminates in an upper grip;
a lower grip assembly including a plurality of sample compartments for receiving the struts, each of the sample compartments configured to hold a sample and having a lower grip affixed to the base of the sample compartment; and
a sleeve enclosing the upper and lower grips for receiving each sample.

27. The bioreactor chamber of claim 26, wherein a volume of medium is circulated through each sample compartment after removal of the sleeve from the sample compartment.

28. The bioreactor chamber of claim 27, wherein the volume of medium contained in each sample compartment is controlled by a plurality of ports.

29. The bioreactor chamber of claim 28, wherein at least one of the ports is configured to receive a plug.

30. The bioreactor chamber of claim 28, wherein the medium is recirculated through the bioreactor chamber.

31. The bioreactor chamber of claim 26, wherein the bioreactor chamber is configured to be received in an enclosure assembly, the bioreactor chamber being enclosed along its length by a plurality of columns supported by a lid and a base.

32. The bioreactor chamber of claim 31, and further including an outer tube or enclosure tube positioned inside the plurality of columns for maintaining the bioreactor chamber in a sterile environment.

33. The bioreactor chamber of claim 26, wherein the plurality of struts are adjustable by raising or lowering a extension rod, thereby raising or lowering each upper grip.

34. The bioreactor chamber of claim 26, wherein the upper and lower grips are wires made from stainless steel.

35. The bioreactor chamber of claim 26, wherein the bioreactor chamber enclosed in an enclosure assembly is a module for being received in an incubator, water bath, a mechanical testing device, or a mechanical stimulation device.

36. The bioreactor chamber of claim 26, wherein the sleeve is configured to slide along the strut.

37. The bioreactor chamber of claim 36, wherein the sleeve is removed from the sample compartment after the sample sets around the upper and lower grips.

38. A method for using a bioreactor chamber, comprising the steps of:
providing a sample compartment configured to hold a sample;
injecting a liquid polymer-cell suspension of the sample into a sleeve contained in the sample compartment, the sleeve enclosing at least a lower grip; and
inserting a strut into the sample compartment, the strut terminating in an upper grip, wherein the sample is arranged to set in the upper and lower grips.

39. The method of claim 36, further including steps of:
removing the sleeve from the sample compartment; and
circulating medium through the sample compartment.

40. The method of claim 39, further including a step of:
providing a plurality of ports to control the circulation of medium through the sample compartment.

41. The method of claim 40, further including a step of:
sampling the medium through at least one of the ports.

42. The method of claim 39, further including a step of:
placing the bioreactor chamber in an incubator, water bath, a mechanical testing device, or a mechanical stimulation device.

43. The method of claim 38, further including a step of:
adding supplements, including but not limited to bioactive factors, proteins, nucleic acids, chemical compounds, and pharmaceuticals through a port of the bioreactor chamber.

44. The method of claim 38, wherein the sleeve is transparent and non-adherent to the sample.

45. The method of claim 44, further including a step of:
UV-curing the sample through the sleeve.

46. The method of claim 44, further including a step of:
thermally curing the sample through the sleeve.

47. The method of claim 46, wherein the step of thermally curing the sample includes placing the bioreactor chamber in a water bath or an incubator.

48. The method of claim 38, wherein the strut is operably connected to a extension rod for adjusting the height of the upper grip.

49. The method of claim 48, further including a step of:
applying uniaxial forces through the extension rod.

50. The method of claim 49, wherein at least one of frequency, duration, magnitude, and waveform can be varied during the step of applying uniaxial forces.

51. The method of claim 38, further including a step of simultaneously applying mechanical loads and medium perfusion.

52. The method of claim 38, further including a step of adding one or more compounds or molecules to the sample compartment to chemically cure the sample.

53. A bioreactor chamber, comprising:
a split mold having a sample compartment configured to hold a sample;
a rod terminating in at least one grip, the at least one grip received in the sample compartment.

54. The bioreactor chamber of claim 53, further including one or more additional sample compartments for holding additional samples.

55. The bioreactor chamber of claim 53, wherein the rod is adjustable to raise or lower the at least one grip in the sample compartment.

56. The bioreactor chamber of claim 53, further including a second grip received in the sample compartment.

57. The bioreactor chamber of claim 53, wherein the split mold is formed by combining a pair of mold halves.

58. The bioreactor chamber of claim 57, wherein the mold halves form a precision seal between them to prevent leakage.

59. The bioreactor chamber of claim 53, wherein the split mold is removable from the bioreactor chamber.

60. The bioreactor chamber of claim 59, wherein a window is attached to the bioreactor chamber after removal of the split mold.

61. The bioreactor chamber of claim 53, wherein a scaffold or material is attached to the at least one grip, the scaffold or material being seeded with cells then the split mold is filled with medium containing cells.

62. The bioreactor chamber of claim 1, wherein a scaffold or material is attached to the at least one grip, the scaffold or material being seeded with cells then the sleeve is filled with medium containing cells.
